(19) United States
(12) Patent Application Publication
(10) Pub. No.: US 2024/0042406 A1
(43) Pub. Date: Feb. 8, 2024

(54) METHOD FOR CARRYING OUT A CHEMICAL REACTION IN AN UPFLOW REACTOR

(71) Applicant: SABIC Global Technologies B.V., Bergen op Zoom (NL)

(72) Inventors: Paulus Johannes Maria Eijsbouts, Nieuwkuijk (NL); Surya Prakasa Rao Daliparthi, Bangalore (IN); Mouli Murukutla, Bangalore (IN)

(21) Appl. No.: 18/266,323

(22) PCT Filed: Dec. 7, 2021
(86) PCT No.: PCT/EP2021/084495

§ 371 Date: Jun. 9, 2023

(30) Foreign Application Priority Data
Dec. 10, 2020 (EP) 20213197.5
Jun. 2, 2021 (EP) 21177285.0

(51) Int. Cl. B01J 8/02; B01J 35/00; B01J 35/02; C07C 37/20; B01J 31/10

(52) U.S. Cl. CPC B01J 8/025; B01J 8/0278; B01J 35/0026; B01J 35/023; C07C 37/20; B01J 31/10; B01J 2208/0092; B01J 2208/00805

(57) ABSTRACT

The present invention relates to a method for carrying out a catalysed chemical reaction using one or more liquid reactants, preferably acetone and phenol, in an upflow reactor comprising feeding at least a portion of said reactants to a bottom section of the reactor positioned below a flow distributor plate, passing said portion through the flow distributor plate, passing said portion through a layer of inert particles positioned above and preferably in contact with said flow distributor plate, passing said portion through a catalyst layer comprising a particulate catalyst, said catalyst layer being positioned above and in contact with said layer of inert particles, wherein the reactants react to form a product stream, collecting said product stream via collecting means positioned above said catalyst layer. The invention also relates to a reactor assembly.

The present invention relates to a method for carrying out a catalysed chemical reaction using one or more liquid reactants in an upflow reactor comprising feeding at least a portion of said reactants to a bottom section of the reactor positioned below a flow distributor plate, passing said portion through the flow distributor plate, passing said portion through a catalyst layer comprising a particulate catalyst wherein the reactants react to form a product stream and collecting said product stream via collecting means positioned above said catalyst layer.

The present invention further relates to a reactor assembly for carrying out such a method.

The present invention in particular relates to a method for the manufacture of bisphenol A based on reacting acetone and phenol.

It is known to carry out chemical reactions in an upflow reactor. For example WO 2018/042375 discloses the use of an upflow reactor for producing a dihydroxy compound as well as to a method for producing a dihydroxy compound. The upflow reactor for producing a dihydroxy compound of WO 2018/042375 comprises a vessel, a catalyst bed disposed in said vessel, a distributor in fluid communication with an inlet through which reactants are introduced to said distributor, said distributor being disposed at a lower end of said vessel and comprising distributor perforation(s) disposed in said distributor, at least part of which distributor perforations are in a direction facing away from said catalyst bed; and a collector through which said product dihydroxy compound is removed, said collector being disposed at an upper end of said vessel.

An upflow reactor for the manufacture of dihydroxy compounds such as bisphenol A is also disclosed in WO 2004/033084.

WO 2020/099285 discloses a method for manufacturing a bisphenol compound comprising reacting a phenol and a ketone in the presence of a catalyst comprising particles having a core and a shell, wherein the shell comprises an ion exchange resin covering the core at least in part and wherein the core has a density that is higher than the density of the ion-exchange resin, wherein the core of the particles has a density of at least 2500 kg/m3.

US 2002/0128531 discloses an oligomerization process for the production of higher aliphatic olefins. In the process, a liquid oligomerization feed stream comprising lighter aliphatic olefins is passed to a reactor vessel. The liquid oligomerization feed stream is transported upwardly in the reactor vessel against gravity through a fixed bed of solid oligomerization catalyst under oligomerization conditions.

WO 97/34688 discloses a reactor system for conducting chemical reactions in which a reactor is operated in an upflow mode with a fixed bed catalyst and randomly distributed reactor packing therein. The reactor system and the process in which it is used exhibit plug flow behaviour and are amenable to employing lightly cross-linked ion exchange resin catalysts.

U.S. Pat. No. 4,067,902 discloses an apparatus and method for first mixing continuously at a prechosen flow rate a less dense fluid with a more dense fluid, the fluids being at least partially immiscible, so as to achieve a constant weight ratio of one fluid to the other per prechosen unit of mixed fluid volume transversely across the path of such mixed fluid followed by charging the resulting mixed fluid simultaneously to a multiplicity of tubes in a tubular reactor under conditions of substantially plug flow. At least one of the fluid is a liquid at all times.

The contents of WO 2020/099285, WO2018/042375 and WO2004/033084 are incorporated herein by reference

An advantage of operating a reactor in upflow, in particular for the manufacture of bisphenols such as bisphenol A is that it may allow for higher throughputs, usually expressed in terms of the weight hourly space velocity (WHSV). The WHSV for downflow reactors is generally about 1.0 for the reason that if a higher pressure is applied on top of the catalyst bed, the bed may compress resulting in a higher pressure drop over the catalyst bed. The WHSV is defined as the weight of feed flowing per unit weight of the catalyst per hour. Thus the WHSV may be defined as ton/hour of feed per ton of catalyst, thus having the unit [1/hr].

The present inventors found that if the flow of reactants through an upflow reactor is increased, the catalyst bed may become fluidized and/or may show uneven flow patterns like channeling of the reactant feed through the catalyst bed. Channeling is a condition of flow wherein portions of the catalyst bed may be short-circuited and not contacted properly by the fluid in a uniform and consistent manner. An uneven flow through the catalyst bed, such as channeling, and/or fluidisation of the same may result in fluctuations of the product mixture composition and generally results in a lower conversion of the reactants and accordingly less efficient use of the catalyst. It was in particular found that distribution plates generate high fluid velocities at or near the openings of such plates. These local fluid velocities are generally higher than the velocity needed for fluidisation while the overall fluid velocity, calculated as the volumetric flow divided by the surface area of the reactor is well below such fluidisation velocity. Thus, even though the overall fluid velocity may not result in fluidisation of the catalyst bed, localised fluidisation occurs and manifests itself for example by means of channeling through or back mixing of the catalyst particles. In case of channeling the feed is less in contact with the actual catalyst thereby adversely affecting the conversion of the reactants and/or the selectivity of the intended product (if applicable). For example, in case of a process to manufacture bisphenol A on the basis of acetone and phenol the channeling will result in a lower acetone conversion and the product mixture may comprise more bisphenol isomers.

It is therefore an object of the present invention to provide for a reactor assembly and a method that allows the operation in an upflow reactor wherein uneven flow and/or channeling is avoided or at least reduced to a minimum.

It is another object of the invention to provide for an upflow reactor assembly and a method wherein the catalyst bed has improved stability and can operate at improved weight hourly space velocities.

The aforementioned objects are met, at least in part in accordance with the invention which relates to a method for carrying out a catalysed chemical reaction using one or more liquid reactants in an upflow reactor comprising:
feeding at least a portion of said reactants to a bottom section of the reactor positioned below a flow distributor plate,
passing said portion through the flow distributor plate,
passing said portion through a layer of inert particles positioned above and preferably in contact with said flow distributor plate,
passing said portion through a catalyst layer comprising a particulate catalyst, said catalyst layer being positioned above and in contact with said layer of inert particles, wherein the reactants react to form a product stream,
collecting said product stream via collecting means positioned above said catalyst layer, wherein,
the upflow reactor is operated at a weight hourly space velocity of at least 1.0, and
the inert particles have a density of at least 2000 kg/m3 and an average particle size of from 500 to 5000 μm, preferably from 500 to 3000 μm, more preferably from 600 to 1500 μm and
the particulate catalyst consists of catalyst particles having a density of at most 65%, preferably at most 50%, of the density of the inert particles and an average particle size, in use, of from 500 to 1500 μm,
the height of the layer of inert particles is at least 40 times the average particle size of the inert particles.

The present inventors found in particular that the openings in a distributor plate may give rise to a locally increased speed of the reactants potentially causing uneven flow patterns, back-mixing and disruptions or channeling in the layer on top of the distributor plate. Accordingly the present invention requires that a layer of inert particles is positioned on top of the distributor plate and between the distributor plate and the catalyst bed or catalyst layer. The function of the layer of inert particles is to homogenise the flow of the reactants as much as possible so that the reactants will move through the catalyst bed in a plug flow mode or at least substantially in plug flow mode. As a result the catalyst bed or catalyst layer stability will be improved and back mixing and channeling through the catalyst layer is reduced to a minimum.

The distributor plate should allow for the reactants to flow in an upwards manner while at the same time it should act as a support for the layer of inert particles. Thus, the openings or holes in the distributor plate should not be too big as otherwise the inert particles may travel through the distribution plate into the bottom section of the reactor. On the other hand, the holes or openings should also not be too small as that would increase the pressure required to pass the reactants through the distributor plate. To the extent the inert particles contain a fraction of fines that may have a size smaller than the openings of the distributor plate this fraction should be kept as low as possible, preferably at most 15 wt. % based on the weight of the layer of inert particles. Preferably the distributor plate is a slotted plate with a plurality of openings having a size smaller than the average particle size of the inert particles and preferably having a size from 50 to 500 μm, preferably from 150 to 300 μm. The distributor plate preferably has a porosity of 10-50%, preferably from 15 -35%, wherein the porosity is defined as the percentage of surface area through which reactants can flow relative to the surface area in flow direction of the distributor plate. Put differently, the porosity refers to the total surface area of openings in the distributor plate relative to the total surface area of such a plate in flow direction.

The inert particles constituting the layer of inert particles are particles having a relatively high density and a preferred (average) particle size. Thus, the present inventors have found it essential that the particles have a density of at least 2000 kg/m3 and an average particle size of from 500 to 5000 μm. The higher the density of the particles, the more resistance they will provide to the flow coming from the distributor plate and the better they are capable of evening out any uneven flow patterns. The present inventors found that particles in the range of from 500 to 5000 μm should be used for the present invention. A preferred average particle size may be from 500 to 3000 μm, more preferably from 600 to 1500 μm. As explained particles with a diameter smaller than the diameter of the holes in distributor plate may migrate to the bottom part of the reactor while particle having a too large particle size become less effective in stabilising the flow of reactants. The particle size distribution may be broad or narrow and may be mono-modal or multimodal such as bimodal. In an embodiment the layer with inert particles is a combination of two or more layers with mutually different average particle size stacked on top of each other, for example with decreasing average particle sizes. The average particle size of the inert particles may be determined using known methods, a sieving method being preferred. It is preferred that the inert particles are obtained by a sieving method so that there is no fraction of particles having a size smaller the openings of the distributor plate. Thus, it is preferred that there is no fraction of inert particles having a size smaller than 500 μm or 600 μm (as the case may be). Put differently it is preferred that at least 95 wt. %, preferably at least 99 wt. %, more preferably at least 99.9 wt. % of the inert particles has a particle size from 500-5000 μm, preferably from 600-3000 μm. The term “particle size” means the diameter in case of spherical particles.

The height of the inert layer in accordance with the invention is at least 40 times the average particle size, typically 40-80, such as 40-60 times the average particle size (diameter) of the inert particles. The present inventors found that this height is sufficient for homogenising the flow patterns and to reduce any local flow deviations to a minimum thereby generating a substantial plug flow mode. In practice the present inventors found that the height of the layer of inert particles is preferably at least 2.0 cm, preferably at least 2.5, more preferably at least 3.0 cm. The upper limit for the height of the bed is less critical although it should not be excessive as that may require a larger reactor and/or the use of more energy to transport the reactant flow through the bed. Typically therefore the height of the inert layer is at most 150 times, preferably at most 100 times the average diameter of the inert particles. In practice a bed height for the inert particles may be from 2.0-15 cm, preferably from 3.0-10 cm. The term plug flow mode in the context of the invention specifically means that the flow-velocity of the reactants is substantially constant over the cross section of the reactor and that there is no or very limited back mixing, wherein the term substantially constant means that the flow-velocity varies at most 5% with respect to the average flow-velocity.

It is preferred that the inert particles comprise, essentially consist or consist of particles selected from sand particles, glass particles, ceramic particles, diatomaceous earth particles, inert metal particles and combinations of at least two hereof. Sand, in particular silica sand, is the preferred material. By means of known sieving techniques the desired particle size and/or particle size distribution for the inert particles can be obtained. The density of the inert particles may be from 2000-5000 kg/m³, preferably from 2100-3500 kg/m³, 2300-3000 kg/m³, 2300-2800 kg/m³. It is preferred that only a single type of material is used, i.e. that the density of each particle, regardless of its particle size, is substantially the same meaning that the density of each particle is at most 10%, preferably at most 5% larger or smaller than the average density for all of the particles.

Alternatively the inert particles may comprise first and second types of materials wherein the first material has an average particle size smaller than an average particle size of the second material and a density that is higher than the density of the second material.

The type of catalyst positioned above and typically in contact with the layer of inert particles is not critical and may be selected for the type of reaction that is to be carried out in the reactor. For the purpose of illustrating the present invention it is preferred that the catalyst is a particulate acidic ion exchange resin type catalyst for the manufacture of bisphenol A. More in particular the catalyst consists of beads on an (acidic) ion exchange resin catalyst. It is particularly preferred that the catalyst beads consist of sulfonated and partially cross-linked polystyrene. The catalyst may contain promotor molecules that are chemically attached to the ion exchange resin allowing for enhancing the selectivity towards the formation of p,p,-bisphenol A. Cross-linked polystyrene based (acidic) ion exchange resin catalysts, including such catalysts containing an attached promotor are known to the skilled person.

The catalyst particles generally have a much lower density compared to the particles of the inert layer. Put differently, the particles of the inert layer have a much higher density compared to the catalyst layer. For the purpose of the invention the catalyst particles have a density of at most 65% of the density of the inert particles and an average particle size, in use, of from 500 to 1500 μm. Preferably the density of the catalyst is from 20-65%, more preferably from 30-60%, even more preferably 35-55% of the density of the inert particles. The density of the catalyst may be from 800-1300 kg/m³, preferably from 1050-1250 kg/m³. For the avoidance of doubt it is noted that the density of the catalyst particles means the overall density of the particles. Accordingly, the core-shell particles as disclosed in WO 2020/099285 have a density that is based on the combination of the (high density) core material and that from the (lower density) shell material even though the shell is the active part of the particles.

The particle size, in use, may be from 600-1200 μm such as from 900-1100 μm. The term “in use” refers to the particle size of the catalyst when it is used in carrying out the intended chemical reaction. In particular ion exchange resins may shrink or expand depending on the reaction medium that they are exposed to and/or depending on the medium in which they are supplied to the end-user. To determine the “in use” diameter a sample of catalyst can be taken from the reactor and analysed using known means for determining the particle size. For the avoidance of doubt it is noted that the present invention is not limited to catalysts that show such shrinkage or expansion. The catalyst may have a particle size distribution and accordingly it is preferred that at least 95 wt.%, more preferably at least 99 wt. %, more preferably at least 99.9 wt. % of the catalyst particles has a particle size from 500 to 1500 μm, preferably from 600-1200 μm. Typical ion-exchange resin catalysts are comprised of substantially spherical beads so that accordingly it is preferred that the average diameter of the catalyst is from 500 to 1500 pm, preferably from 600-1200 μm. Likewise it is preferred that at least 95 wt. %, more preferably at least 99 wt. % or 99.9 wt. % of the catalyst particles has a diameter from 500 to 1500 μm, preferably from 600-1200 μm.

The present invention allows a chemical reaction to be carried out at a higher weight hour space velocity (WHSV). The WHSV is defined as the weight of feed flowing per unit weight of the catalyst per hour. The present invention allows chemical reactions to be carried out at higher WHSV as compared to down-flow reactors while maintaining a desired conversion of the reactants. That is the present invention allows for a chemical reaction to take place at a WHSV of at least 1.0, such as at least 1.1 or at least 1.2. Preferably the WHSV is from 1.1-5, 1.2-4, 1.5-3.5 more preferably from 1.7-3.

The present invention is preferably directed at a method for the manufacture of a bisphenol, in particular bisphenol A, by reacting ketone with phenol. More specifically the present invention is directed at a method for the manufacture of bisphenol A by reacting acetone and phenol in the presence of an ion-exchange resin catalyst. In such method the product stream comprises bisphenol A, phenol, acetone, water and by products, wherein the amount of bisphenol A is from 15-35, preferably 20-30 wt. % based on the weight of the product mixture. Likewise the present invention is preferably directed at a reactor assembly for the manufacture of bisphenol A by reacting acetone and phenol in the presence of an (acidic) ion-exchange resin catalyst.

The present invention also relates to a reactor assembly for carrying out a catalysed chemical reaction, such as the method disclosed herein in all its aspects and preferences, using one or more liquid reactants in upflow comprising:
feeding means for feeding at least a portion of said reactants to a bottom section of the reactor, said feeding means being positioned below a flow distributor plate,
a layer of inert particles positioned above and preferably in contact with said flow distributor plate,
a catalyst layer comprising a particulate catalyst positioned above and in contact with said layer of inert particles,
collecting means positioned above said catalyst layer for collecting a product stream from the reactor,
wherein,
the inert particles have a density of at least 2000 kg/m3 and an average particle size of from 500 to 5000 μm, preferably from 500 to 3000 μm, more preferably from 600 to 1500 μm, and
the particulate catalyst consists of catalyst particles having a density of at most 65%, preferably at most 50% of the density of the inert particles and an average particle size, in use, of from 500 to 1500 μm,
the height of the layer of inert particles is at least 40 times the average particle size of the inert particles.

The reactor used for the method and the apparatus disclosed herein is typically substantially cylindrical in shape. Substantially cylindrical means that the ratio between the largest diameter and the smallest diameter is from 0.9-1.1.

For the avoidance of doubt it is to be understood that the present invention relates to a method and reactor assembly for carrying out one or more chemical reactions in a liquid phase. Thus, the present method excludes gas-phase reactions. Although the general principle of the reactor assembly will work for gas phase reactions as well the requirements for WHSV, catalyst density, catalyst particle size, inert particle density and inert particle size will be different as will be understood by a skilled person.

All preferred aspects disclosed herein related to the process apply also to the reactor assembly, if applicable.

The present invention will now further elucidated herein on the basis of the following non-limiting examples and Figures.

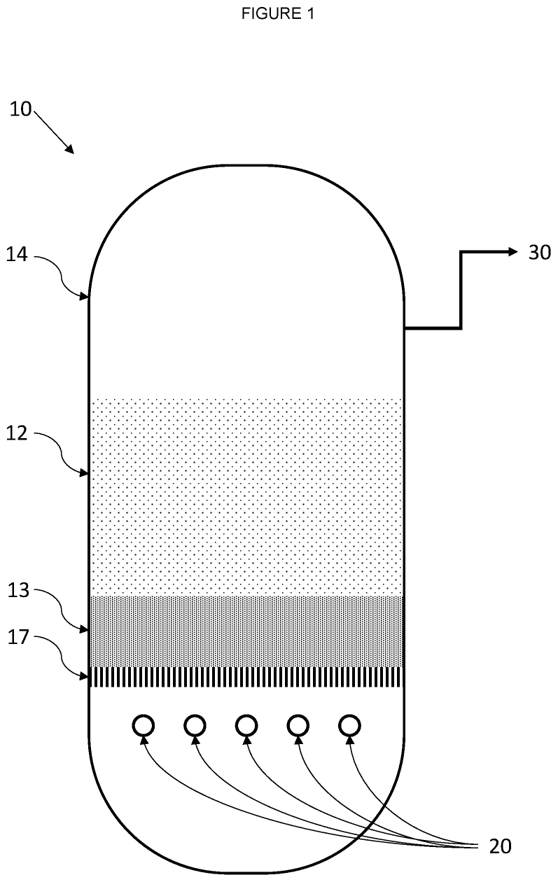

FIG. 1, not to scale, is a partial cross-sectional view of an exemplary reactor assembly 10 of the present disclosure. In general, reactor assembly 10 is a packed bed reactor assembly that is configured and dimensioned to produce dihydroxy compounds (e.g., bisphenols, preferably bisphenol A). Reactor assembly 10 includes a distributor unit 20 having a plurality of distributor pipes (e.g., four distributor pipes) with holes or perforations pointing downward, with the plurality of distributor pipes 20 forming a distributor unit positioned evenly across the cross-section of the vessel 14 of reactor assembly 10.

Above the distributor unit, a distributor plate 17 (e.g., a slotted plate or wire mesh plate with openings having a size from 50 to 500 micrometers, preferably from 150 to 300 micrometers) is positioned to support the layer of inert particles 13 and the catalyst layer 12. The feed to the reactor assembly 10 via distributor pipes 20 of the distributor unit includes phenol and acetone. From the pipes 20 the liquid feed is injected predominantly in a direction away from the distributor plate 17.

The inert layer, i.e. layer with inert particles 13 is positioned on or above distributor plate 17 to help distribute the feed from the distributor unit uniformly to the catalyst layer 12.

The inert layer 13 can comprise, without limitation, sand particles, glass particles, silica particles, metal particles (e.g., metal particles that are inert in the feed mixture, such as, for example, nickel particles and/or titanium particles), or a combination comprising at least one of the foregoing. The height of the inert layer 13 (e.g., sand layer 13) on top of distributor plate 17 in vessel 14 can be on average 2.5 cm.

A lower limit for the height of the inert layer 13 on top of distributor plate 17 can be about 2 cm. However, it is noted that the height of the layer 13 on top of distributor plate 17 can be 4 cm to 5 cm. It is noted that there may be no significant benefit having a height of the layer 13 on top of distributor plate 17 greater than 10 cm.

The particles (e.g., sand particles) of layer 13 can have an average particle size from 700 to 1200 μm.

In use, a catalyst layer 12 can be disposed on the inert layer 13 (layer of inert particles), said catalyst layer 12 including ion exchange resin particles (e.g., acidic ion exchange resin catalyst particles), the ion exchange resin particles optionally comprising an attached promoter (e.g., co-catalyst promoter).

For the avoidance of doubt it is noted that the flow direction in reactor assembly 10 is from bottom to top , i.e. from distributor pipes 20 to collecting means 30.

Collecting means 30 for collecting the product mixture from reactor assembly 10 are positioned above catalyst layer 12. The exact location of said collecting means may vary.

In an embodiment a further product distribution plate (not shown) may be positioned above catalyst layer 12. Such a product distribution plate is optionally in direct contact with the catalyst layer. The porosity of such a distribution plate is preferably the same or higher than the porosity of the distributor plate. Preferably the product distributor plate is a slotted plate with a plurality of openings having a size smaller than the average particle size of the catalyst particles and preferably having a size from 300-1000 μm, preferably from 500-800 μm. The product distributor plate, in particular when in contact with the catalyst layer 12 further supports the catalyst bed stability and may be used to prevent catalyst particles to end up in the collecting means. A combination of several product distributor plates may be used.

The product mixture typically contains bisphenol A, acetone, phenol, water and impurities or byproducts. The product mixture is further processed in downstream process steps to isolate and purify the bisphenol A product. Such steps are known to a skilled person.

In a specific aspect the present invention relates to a method for carrying out a catalysed chemical reaction for the manufacture of bisphenol A using one or more liquid reactants in an upflow reactor comprising:
feeding at least a portion of said reactants to a bottom section of the reactor positioned below a flow distributor plate, said reactants comprising acetone and phenol,
passing said portion through the flow distributor plate,
passing said portion through a layer of inert particles positioned above and preferably in contact with said flow distributor plate,
passing said portion through a catalyst layer comprising a particulate acidic ion exchange resin catalyst optionally comprising an attached promotor, said catalyst layer being positioned above and in contact with said layer of inert particles, wherein the reactants react to form a product stream comprising bisphenol A,
collecting said product stream via collecting means positioned above said catalyst layer,
wherein,
the upflow reactor is operated at a weight hourly space velocity of at least 1.0, and
the inert particles have a density of at least 2000 kg/m3 and an average particle size of from 500 to 5000 μm preferably from 500 to 3000 μm, more preferably from 600 to 1500 μm, and
the particulate catalyst consists of catalyst particles having a density of from 800 to 1200 kg/m³ and an average particle size, in use, of from 500 to 1500 μm,
the height of the layer of inert particles is at least 40 times the average particle size of the inert particles.

The preferred features as described herein more generally equally apply to the method of this specific aspect.

### EXAMPLES 1-7

Experiments were conducted using the configuration of the reactor assembly 10 as shown in FIG. 1. Reactor assembly 10 included a distributor unit having four distributor pipes 20 with holes or perforations pointing downward, with the four distributor pipes of distributor unit 20 positioned evenly across the cross-section of vessel 14.

Vessel 14 had an inner diameter of 800 millimeters (mm). The tangent to tangent height of vessel 14 of 2000 mm was sufficient to contain the catalyst volume of catalyst layer 12 and provided sufficient empty space for proper liquid collection using suitable collecting means 30. Above the distributor unit, a distributor plate 17 was positioned. Distributor plate 17 was a slotted plate with openings having a diameter of 200 μm.

An amount of 0.50 m 3 of ion-exchange resin catalyst formed catalyst bed 12.

The ion-exchange resin catalysts for catalyst bed 12 used in the experiments were commercially available 2% cross-linked sulfonated polystyrene which differed in particle size distribution.

Catalyst Type 1 had a poly-disperse particle size distribution with a ratio of D90/D10 of about 1.4 and with an average particle size of about 1065 μm.

Catalyst Type 2 had a mono-disperse particle size distribution with a ratio of D90/D10 of about 1.1 and with an average particle size of about 875 μm.

The particle size and particle size distribution was determined via an image analysis technique.

Table 1 shows results of experiments carried out at different flow speeds, representative for the amount of material flowing through the reactor and accordingly for the WHSV. The feed consisted of a mixture of 5 wt. % acetone and 95 wt. % phenol and catalyst type 2 was used. The sand layer in examples E1-E3 consisted of sand particles obtained by sieving of river sand and having a particle size distribution from 0.7 to 1.2 mm, and a bulk density of 1.58 kilogram/liter (kg/l). The height of the sand layer 13 on top of distributor plate 17 in vessel 14 was on average 2.5 cm. The average particle size of the sand particles was about 1000 μm. The density of the sand particles of sand layer 13 was about 2500 kg/m³.

TABLE 1

| | E1 | E2 | E3 | CE1 | CE2 | CE3 |
|---|---|---|---|---|---|---|
| Flow_speed [mm/s] | 0.133 | 0.200 | 0.267 | 0.133 | 0.200 | 0.267 |
| WHSV | 1.0 | 1.5 | 2.0 | 1.0 | 1.5 | 2.0 |
| Acetone conversion [%] | 94.5 | 89.6 | 74.5 | 96.3 | 85.9 | 71.8 |
| ppBPA Selectivity [%] | 94.0 | 93.7 | 93.7 | 94.0 | 94.6 | 94.0 |
| Feed temperature [° C.] | 55 | 55 | 60 | 55 | 55 | 60 |
| Sand layer | yes | yes | yes | no | no | no |
| Catalyst type | 2 | 2 | 2 | 2 | 2 | 2 |

The flow speed corresponds to the upward linear velocity of the flow in the void reactor area (e.g., the area above catalyst bed 12) of vessel 14 and is defined as the volumetric flowrate divided by the cross-sectional area of the vessel 14 of the reactor assembly 10.

The acetone conversion represents the weight percent of acetone that is converted during the reaction and based on measurement of the acetone concentration in the outlet of the reactor.

The p,p-bisphenol A (ppBPA) selectivity represents the weight percentage of p,p-bisphenol A that was produced relative to the total amount of bisphenols.

From Table 1 it can be observed that upon increasing flow speed the acetone conversion decreases when no sand bed (layer of sand) is present. The present inventors believe this was due to an uncontrolled and less homogenous flow through the catalyst layer and may either one or more of catalyst fluidisation and/or back mixing and/or channeling.

Table 2 shows the results of experiments that were carried under the same conditions except that catalyst type 1 was used and the concentration of acetone in the feed was 3 wt. % (and the phenol concentration accordingly was 97 wt. %)

TABLE 2

| | E4 | E5 | CE4 | CE5 |
|---|---|---|---|---|
| Flow_speed [mm/s] | 0.133 | 0.267 | 0.133 | 0.267 |
| WHSV | 1.0 | 2.0 | 1.0 | 2.0 |
| Acetone conversion [%] | 98.2 | 93.1 | 96.2 | 88.7 |
| ppBPA Selectivity [%] | 95.1 | 95.9 | 945.4 | 94.7 |
| Feed temperature [° C.] | 55 | 55 | 55 | 55 |
| Sand layer | yes | yes | no | no |
| Catalyst type | 1 | 1 | 1 | 1 |

Table 3 shows the results of further experiments wherein the feed contained fresh acetone and phenol but also a recycle stream containing unreacted acetone and phenol, p,p-bisphenol-A (also known as 2,2-bis(4-hydroxyphenyl)propane or “ppBPA”)), o,p-bisphenol A (also known as 2,4′-isopropylidenediphenol (“opBPA”)) and/or other isomers. Catalyst type 1 was used; the sand layer (if present) was the same as for the Examples in Tables 1 and 2.

The composition of the feed in the experiments E6/CE6 and E7/CE7 consisted of
3 wt. % of acetone
74.5 wt.% of phenol
12 wt. % of p,p-bisphenol A
3.5 wt. % of o,p-bisphenol A
7 wt. % other isomers including one or more of, 3-(4-hydroxyphenyl)-1,1,3-trimethyl-2H-inden-5-ol (“cyclic dimer 1”); 2,4-bis[1-(4-hydroxyphenyl)isopropyl]phenol (“BPX 1”); 4-(2,2,4-trimethylchroman-4-yl)phenol (“chroman 1”); 4-(2,4,4-trimethyl-3,4-dihydro-2H-chromen-2-yl)phenol (“chroman 1.5”); 1,1′-spirobi[1H-indene]-6,6′-dio1,2,2′,3,3′-tetrahydro-3,3,3′,3′-tetramethyl (“spirobiindane”).

The viscosity of this feed was significantly higher compared to the feed in the experiments in Tables 1 and 2.

TABLE 3

| | E6 | E7 | CE6 | CE7 |
|---|---|---|---|---|
| Flow_speed [mm/s] | 0.133 | 0.267 | 0.133 | 0.267 |
| WHSV | 1.0 | 2.0 | 1.0 | 2.0 |
| Acetone conversion [%] | 95.2 | 65.2 | 77.1 | 44.6 |
| ppBPA Selectivity [%] | 98.4 | 98.8 | 101.2 | 103.2 |
| Feed temperature [° C.] | 62.8 | 63.2 | 56 | 63.4 |
| Sand layer | yes | yes | no | no |
| Catalyst type | 1 | 1 | 1 | 1 |

Because of the presence of isomers in the feed, which may be converted into p,p-bisphenol A, the ppBPA selectivity could be over 100%.

Since the feed in the Examples of Table 3 has a significantly higher viscosity (about 2.5 times higher) compared to the Examples in Tables 1 and 2, the settling rate of the catalyst particles in the feed medium was higher, explaining a larger effect on the acetone conversion. Additionally and as mentioned, because this feed also contains products that can be converted by the catalyst into ppBPA (e.g., isomerisation of o,p-bisphenol A) the net calculated ppBPA selectivity % can be above 100%.

By means of computational fluid dynamics simulations the present inventor confirmed that advantageous effect of the sand layer. For a reactor equipped with a tri-slot distributor plate having 1.8 mm width bars with 0.2 mm clearance between them they found that at a WHSV of 2 they observed that without a sand layer significant channeling and/or back mixing occurred in the catalyst layer. When the catalyst layer was put on top of a sand layer of about 5 cm the channeling and back mixing was reduced to a minimum and a more even fluid velocity pattern was observed.

In the claims:

1. Method for carrying out a catalysed chemical reaction using one or more liquid reactants in an upflow reactor comprising:
feeding at least a portion of said reactants to a bottom section of the reactor positioned below a flow distributor plate,
passing said portion through the flow distributor plate,
passing said portion through a layer of inert particles positioned above and in contact with said flow distributor plate,
passing said portion through a catalyst layer comprising a particulate catalyst, said catalyst layer being positioned above and in contact with said layer of inert particles, wherein the reactants react to form a product stream,
collecting said product stream via collecting means positioned above said catalyst layer,
wherein,
the upflow reactor is operated at a weight hourly space velocity of at least 1.0, and
the inert particles have a density of at least 2000 kg/m3 and an average particle size of from 500 to 5000 μm, and
the particulate catalyst consists of catalyst particles having a density of at most 65% of the density of the inert particles and an average particle size, in use, of from 500 to 1500 μm,
the height of the layer of inert particles is at least 40 times the average particle size of the inert particles.

2. The method of claim 1 wherein the catalysed chemical reaction comprises the reaction between phenol and ketone, so as to form a product stream comprising bisphenol.

3. The method of claim 1 wherein the weight hourly space velocity is at least 1.2.

4. The method claim 1 wherein the liquid reactants consist of phenol and acetone, the product stream comprises bisphenol A, phenol, acetone, water and by products, wherein the amount of bisphenol A is from 15-35 wt. % based on the weight of the product mixture.

5. The method of claim 1 wherein the inert particles comprise, essentially consist or consist of particles selected from sand particles, glass particles, ceramic particles, diatomaceous earth particles, inert metal particles and combinations of at least two hereof.

6. The method of claim 1 wherein the catalyst is a cross-linked polystyrene based ion-exchange resin type catalyst.

7. The method of claim 1 wherein the distributor plate is a slotted plate with a plurality of openings having a size smaller than the average particle size of the inert particles and preferably having a size from 50 to 500 μm.

8. The method of claim 1 wherein the distributor plate has a porosity of 10-50%, porosity being defined as the percentage of open area relative to the surface area in flow direction of the distributor plate.

9. The method of claim 1 wherein the height of the layer of inert particles is at least 2.0 cm.

10. The method of claim 1 wherein the reactor is substantially cylindrical in shape.

11. The method of claim 1 wherein the liquid reactants consist of phenol and acetone, the product stream comprises bisphenol A, phenol, acetone, water and by products, wherein the amount of bisphenol A is from 20-30 wt. % based on the weight of the product mixture.

12. A reactor assembly for carrying out a catalysed chemical reaction using one or more liquid reactants in upflow comprising:
feeding means for feeding at least a portion of said reactants to a bottom section of the reactor, said feeding means being positioned below a flow distributor plate,
a layer of inert particles positioned above and in contact with said flow distributor plate,
a catalyst layer comprising a particulate catalyst positioned above and in contact with said layer of inert particles,
collecting means positioned above said catalyst layer for collecting a product stream from the reactor,
wherein,
the inert particles have a density of at least 2000 kg/m3 and an average particle size of from 500 to 5000 μm, and
the particulate catalyst consists of catalyst particles having a density of at most 65% of the density of the inert particles and an average particle size, in use, of from 500 to 1500 μm,
the height of the layer of inert particles is at least 40 times the average particle size of the inert particles.

13. The reactor assembly of claim 12 for carrying out the method in accordance with claim 1.

14. The method of claim 1, wherein the particulate catalyst consists of catalyst particles having a density of at most 50% of the density of the inert particles.

15. The method of claim 1, wherein the catalysed chemical reaction comprises the reaction between phenol and acetone so as to form a product stream comprising bisphenol A.

16. The method of claim 1, wherein weight hourly space velocity is from 1.5 to 5.0, or from 1.7 to 3.0.

17. The method of claim 1, wherein the catalyst is a cross-linked polystyrene based ion-exchange resin type catalyst and comprises an attached promotor.

18. The method of claim 1, wherein the distributor plate is a slotted plate with a plurality of openings having a size smaller than the average particle size of the inert particles and having a size from 150 to 300 μm.

19. The method of claim 1, wherein the height of the layer of inert particles is at least 2.5 cm.

20. The method of claim 1, wherein the height of the layer of inert particles is at least 3.0 cm.
